(19) United States
(12) Patent Application Publication
(10) Pub. No.: US 2022/0151275 A1
(43) Pub. Date: May 19, 2022

(54) Dietary Supplement for Gastrointestinal Inflammation and Method for Making the Same

(71) Applicant: CS Health Solutions, LLC, Highland Park, IL (US)

(72) Inventors: James L. Babb, Libertyville, IL (US); James J. Koziarz, Highland Park, IL (US); Raymond J. Laudano, Libertyville, IL (US)

(21) Appl. No.: 17/587,704

(22) Filed: Jan. 28, 2022

Related U.S. Application Data
(63) Continuation of application No. 15/876,098, filed on Jan. 19, 2018.
(60) Provisional application No. 62/448,151, filed on Jan. 19, 2017.

(51) Int. Cl. A23L 33/10; A61K 35/38

(52) U.S. Cl. CPC A23L 33/10; A61K 35/38; A23V 2002/00; A23V 2200/32; A23V 2250/204; A23V 2300/14; A23V 2250/082

(57) ABSTRACT

A dietary supplement for improving gastrointestinal functionality includes: an edible supplement comprising at least 10 mg/mL of alcohol soluble components of an animal broth. A method for preparing a dietary supplement for improving gastrointestinal functionality includes the steps of: transforming an animal broth into an active agent by adding a transforming vehicle to the animal broth, wherein the transforming vehicle is an alcohol; isolating the active agent by removing precipitates; removing the transforming vehicle from the active agent; and forming the dietary supplement, wherein the dietary supplement includes the active agent.

### CROSS-REFERENCE TO RELATED APPLICATIONS

This application comprises a continuation application of U.S. application Ser. No. 15/876,098 filed Jan. 19, 2018, which claims the benefit of priority to U.S. Provisional Patent Application 62/448,151 filed on Jan. 19, 2017, the entireties of which are incorporated by reference.

### BACKGROUND OF THE INVENTION

The present subject matter relates generally to a dietary supplement, preparation, and method of delivery wherein the supplement promotes an anti-inflammatory effect or a barrier-promoting effect on a human (or another mammalian) gastrointestinal (GI) tract. Specifically, the present subject matter provides dietary supplements comprising an alcohol soluble fraction of meat broths and/or stocks, and their equivalents, wherein the supplement repairs, protects, and/or strengthens an epithelial cell layer in at least an in vitro assay for tight junction formation. Further, the present subject matter provides processes for making the supplements, including heat extracting a source tissue followed by alcohol treatment, as well as processes for alcohol treatment of available broths and stocks.

In human physiology, there are two main types of epithelia, and each uses a distinct type of barrier mechanism. One epithelium is the Dermal structure, such as skin, that forms a barrier comprising multiple layers of keratinised squamous cells. The other is referred to as Internal epithelia, which is what lines the gut. Tight junctions create a paracellular barrier in epithelial cells, protecting them from the external environment. Tight junctions hold cells together and mediate the barrier function.

The important role of the gut epithelial membrane in promoting and maintaining homeostasis in a healthy host is well-established and documented in the scientific literature. This natural barrier aids in important physiological functions such as nutrient uptake from the lumen into the host as well as serving as a sentry to keep toxic substances or pathogens from entering into the host. There are ample examples showing that the gut mucosa is in a constant state of flux characterized by breakdown, repair and renewal. Many publications document the structure function aspect of these processes and illustrate the important features and mechanisms involved in gut epithelial wound healing.

Individuals with GI tract disorders typically demonstrate impaired mucosal barrier function and disrupted tight junctions. Further, the scientific literature documents that many diseases are caused by or exacerbated by an underlying breakdown of this barrier or disruption of normal physiology. For example, oral mucositis, is an inflammatory and ulcerative injury of the mouth, throat, or GI tract most commonly caused by chemo or radiation therapy for cancer. This condition has its onset upon the disruption of the mucosal surface of the mouth and other portions of the GI tract. Both the epithelial layer and the underlying connective tissue of the oral cavity are affected. Cell death occurs and there is a loss of tight junction proteins, such as occludin and ZO-1, at the molecular level. Consequently, there is a subsequent disruption of the tight junctions.

Upon injury, the intestinal epithelium undergoes a wound healing process. Intestinal wound healing is dependent on the balance of three cellular events; restitution, proliferation, and differentiation of epithelial cells adjacent to the wounded area. Various regulatory peptides, including growth factors like gastrokine 1 (AMP-18) and cytokines, modulate intestinal epithelial wound healing. As healing occurs, new and enhanced formation of tight junctions between cells occur. With time, mucosal barrier function is restored.

Mucosal nutrients and growth factors like gastrokines are intermediary or end products of prokaryotic and eukaryotic metabolism. Tissues rich in metabolic activity generally have higher concentrations of the active compounds. Growth factors, structural proteins, and enzymes are synthesized from precursor molecules that have their own biochemical properties. Some may have biological activity, whereas others may require an “activation” cleavage or addition. Little is known about ratios of precursor to active molecules that are required to achieve optimal health. Most nutritional treatment protocols, regimens, and intervention strategies employ supplementation of one or more agents to the diet and rely on the host to provide other complementary factors to restore normal homeostasis.

Nutrients such as L-glutamine, phosphatidylcholine, N-acetyl-D-glucosamine, and gamma-linolenic acid provide the fuel and building blocks used in repair. Their importance is suggested from studies showing that patients having gastrointestinal disorders either lack or have depressed levels of these nutrients in their gut secretions. Investigations are beginning to reveal that suppressed repair mechanisms may lead to more serious diseases, such as the case of down regulated AMP-18 genes in patients with gastric cancer.

Restoration of normal healthy function can be facilitated by traditional medicinal therapies, nutritional supplements, diet, or probiotics. There is no consensus on the optimal levels of the above-mentioned nutrients and growth factors in the gut milieu that maintain a healthy GI tract. However, improved mucosal health has been reported in humans after daily ingestion of vitamins and other nutrients at dosage levels reported to be from 100 mg/day up to 1500 mg/day. These studies also demonstrate that, through oral administration, these compounds are directly absorbed from the gut lumen and incorporated into glycans and glycoproteins of the intestinal mucosa.

The efficacy of gastrokines to treat gastrointestinal inflammation in animal models was achieved at dosages ranging from 1 mg/kg to 100 mg/kg. The route of administration (specifically intraperitoneal or subcutaneous injections, as well as topically with no carrier) did not seem to affect efficacy. However, dose response curves imply a minimum dosage threshold to achieve desired outcomes.

The plethora of options at the local health stores suggests that non-medicinal products can afford benefits, Unfortunately, not all of these alternative therapies have been evaluated in well-control population studies.

Accordingly, there is a need for a dietary supplement that can be shown in well-controlled studies, to be efficacious in aiding in protection and strengthening of the gastrointestinal tract and repair of gastrointestinal inflammation-associated injury. In addition, there is a need for dietary components and mixtures of nutrients that are effective in improving intestinal barrier function without the requirement of pharma grade agents. Lastly, there is a need for a cost-effective and efficient process for preparing a dietary supplement, particularly directly from a biological source.

### BRIEF SUMMARY OF THE DESCRIPTION

The underlying principal to the subject matter described herein is that natural, generally recognized as safe (“GRAS”), food sources can be used to prepare a mixture of agents that can fortify normal physiological wound healing processes and strengthen/reinforce tight junction barrier function. In a primary example, the present disclosure includes a composition comprising an edible food product derived from a combination of hydrolyzed animal tissue, wherein the initial source material is a commercially available chicken broth. An effective composition includes the alcohol soluble components of the initial food source adjusted to appropriate levels. No pharma grade chemicals or synthetics are required or needed. Further, the action of these natural agents can be studied and measured for efficacy and potency in experimental models typically used for the evaluation of pharma products.

The primary examples of the subject matter taught herein relate to supplements for improving human GI tract health and integrity. However, it is understood that the teachings herein may be used for improving the GI tract health and integrity in mammalian animals, specifically, livestock and/or domestic pets.

The term singular and plural versions of the term “agent” (i.e., agent and agents) are used interchangeably throughout this disclosure. Particularly, it is recognized that, as used herein, an active agent may consist of one or more agents. Accordingly, any use of the term agent or agents should be recognized as being inclusive of both singular and plural embodiments and the specific version of the word used in any given context has simply been chosen for simplified clarity in the description.

In its development, the subject matter provided herein was tested for its effect on tight junctions. Tight junctions were chosen to study because of their importance in gut health and the availability of well-controlled in vitro models. The subject matter disclosed herein teaches that a composition of alcohol soluble agents derived from GRAS food substances can interact with tight junctions to enhance their function to protect against inflammatory induced injury and promote a prolonged barrier function greater than that observed in control conditions.

To this end, the present disclosure provides dietary supplement compositions and methods of making the same. In certain embodiments, the supplement compositions include alcohol soluble fractions of animal broths, or their equivalents, that strengthen, protect, and repair epithelial cell layers in (at least) an in vitro assay for tight function formation. Further, the present subject matter provides processes for making the supplements, including heat extracting a source tissue followed by alcohol treatment, as well as processes for alcohol treatment of commercially available broths and stocks.

The supplement compositions are based on the premise that unimpeded wound healing occurs when there are both growth factors and wound healing proteins available at concentrations, and in a format, that favor immediate recruitment. In examples provided herein, such compositions can be achieved by careful selection of proper starting materials, a gentle disruptive hydrolysis, and selective transformation of the heat digest with ethanol. The methods can include activating the broth solution with alcohol treatment, followed by sequestration of the active agent solution by phase separation techniques (e.g., removing any resulting precipitates from the active agent solution).

Specifically, the disclosed supplement composition can be derived from a GRAS natural food source, such as a digest of animal tissue containing a complex mixture of proteins, peptides, growth factors, and nutrients associated with healthy tight junctions and homeostasis. For example, the natural sources may include animal muscle tissue, such as stomachs, livers, and gizzards from chicken, bovine, krill, and sea urchins, among others. One specific example of such a GRAS natural food source is chicken gizzards. Another example of such a GRAS natural food source is commercially available chicken broth.

In a primary example, the starting source material (e.g., animal muscle tissue) is minced into small pieces, converted into a puree (or smooth paste), or subject to any other particle reduction mechanism that increases the surface area of the source material, and agents are released by a gentle heat or equivalent digestion. Soluble extracted agents are harvested by filtration followed by treatment with a transforming vehicle, such as, but not limited to, ethanol, to initiate flocculation and precipitation of insoluble components. The amount of transforming vehicle used can be varied to achieve optimal levels of enrichment to form the active agents. Transformation can be monitored visually or through the use or instrumentation. Alcohol soluble agents are decanted and combined. The transforming vehicle is removed by applying gentle heat and through evaporation. The active agents can be selectively harvested and processed into a liquid or dried supplement for delivery to consumers.

The present disclosure provides a composition for improving gastrointestinal function comprising a mixture of edible food products derived from hydrolyzed animal smooth and/or skeletal muscle tissue, bones, or ligaments. In an example, the composition includes alcohol soluble components adjusted to functional concentrations. The composition can include agents with gastrokine activity.

The disclosure further includes methods for preparation of a dietary supplement for improving gastrointestinal disorders, wherein the method comprises selecting a biological source, applying heat to the biological source to release a biological agent from the biological source, extracting the biological agent from the source to form an extracted agent, transforming the extracted agent with a food grade alcohol, removing the alcohol, and forming the dietary supplement using the remaining alcohol soluble components, wherein the dietary supplement includes the transformed active agent.

In an example of a dietary supplement for improving gastrointestinal functionality according to the teachings provided herein, the dietary supplement includes: a concentrated animal broth derived from an animal source, wherein the concentrated broth is activated by a transforming, wherein the transforming vehicle is an alcohol. In some examples, the transforming vehicle is removed from the dietary supplement. The dietary supplement may be in the form of at least one of a food product, hydrogel, solution, tablet, lozenge, vitamin, powder, candy, and gel.

In another example of a dietary supplement for improving gastrointestinal functionality according to the teachings provided herein, the dietary supplement includes at least 10 mg/mL of alcohol soluble components of an animal broth. More specifically, the dietary supplement may include at least 10 mg of protein derived from the alcohol soluble components of an animal broth per mL of culture media. The protein measurement may be used as a surrogate measurement for the concentration of the active agent in the dietary supplement. For example, the dietary supplement may be provided in powdered form wherein there is at least 10 mg of protein per mL of powder. In liquid preparations, the dietary supplement may be adjusted, for example, to be at least 10% volume of concentrated protein solution to overall preparation volume. It is understood that these are merely illustrative examples of the dietary supplement composition and that other variations will be apparent to those skilled in the art based on the teachings provided herein.

The dietary supplement may be in the form of at least one of a food product, hydrogel, solution, tablet, lozenge, vitamin, powder, candy, and gel. The dietary supplement may be derived from commercially available chicken broth. In another example, the dietary supplement may be derived from an animal broth including one or more chicken gizzards. In some embodiments, the dietary supplement is prepared by: transforming an animal broth into an active agent by adding a transforming vehicle to the animal broth, wherein the transforming vehicle is an alcohol; isolating the active agent by removing precipitates; removing the transforming vehicle from the active agent; and forming the dietary supplement, wherein the dietary supplement includes the active agent.

In one example, a method for preparing a dietary supplement for improving gastrointestinal functionality, wherein the method includes: selecting a biological source; applying heat to the biological source to release a biological agent from the biological source; isolating the biological agent from the biological source to form an isolated agent; transforming the isolated agent into an active agent by treating the extracted agent with an alcohol solution; and forming the dietary supplement using the alcohol soluble components, wherein the dietary supplement includes an effective dose of the active agent.

In certain embodiments of the method, the step of transforming the extracted agent into an active agent includes combining the extracted agent with an ethanol solution, decanting the supernatant, and concentrating the supernatant solution to form the active agent. Isolating the biological agent from the biological source to form an extracted agent may include filtering the biological source from the biological agent. In some examples, the method further comprises, after transforming the extracted agent into the active agent, heating the active agent to reduce the alcohol content. In some embodiments, the biological source is an animal protein. For example, the biological source may be one or more chicken gizzards. In other examples, the biological agent may be commercially available chicken stock.

The method may further include the step of incorporating the dietary supplement into at least one of a food product, hydrogel, tablet, lozenge, vitamin, powder, liquid, candy, and gel. Within the method, the step of transforming the extracted agent into an active agent by treating the extracted agent with an alcohol solution includes adding an amount of alcohol to the active agent in a 1:1 ratio by volume.

In other examples, a method for preparing a dietary supplement for improving gastrointestinal functionality, wherein the method includes: providing an animal broth; transforming the animal broth into an active agent by adding a transforming vehicle to the animal broth, wherein the transforming vehicle is an alcohol; isolating an active agent by removing precipitates; removing the transforming vehicle from the active agent; and forming the dietary supplement, wherein the dietary supplement includes the active agent. In some embodiments, the alcohol is ethanol.

An object of the subject matter of the present disclosure is to provide a dietary supplement that promotes an anti-inflammatory effect or barrier-promoting effect on the human gastrointestinal tract.

Another object of the subject matter of the present disclosure is to provide a dietary supplement that restores and/or improves an epithelial cell membrane integrity.

Another object of the subject matter of the present disclosure is to provide methods of preparation of such supplements.

Another object of the subject matter of the present disclosure is to provide methods of delivery of such supplements.

Another object of the subject matter of the present disclosure is to provide dietary supplements including an alcohol soluble fraction of meat broths and/or stocks, and their equivalents, wherein the supplement strengthens, protects, and repairs an epithelial cell layer in at least an in vitro assay for tight junction formation.

Another object of the subject matter of the present disclosure is to provide processes for heat extracting a source tissue followed by alcohol treatment and to provide processes for alcohol treatment of available broths and stocks.

An advantage of the subject matter of the present disclosure is that it provides supplements that promotes an anti-inflammatory effect or barrier-promoting effect on the human gastrointestinal tract.

Another advantage of the subject matter of the present disclosure is to provide a treatment method using natural food products without additional synthetic drugs that may have adverse side effects.

Another advantage of the subject matter of the present disclosure is that it provides a supplement that has nutritional value.

A further advantage of the subject matter of the present disclosure is that it provides a method of preparation that provides a supplement that does not require dilution and is compatible with a variety of common food and beverage ingredients to yield a desired flavor and texture profile.

Another advantage of the subject matter of the present disclosure is the fortification of normal wound healing processes using natural agents (i.e., not chemically synthesized) useful in maintaining gastrointestinal health and addressing gastrointestinal disorders.

Yet another advantage of the subject matter of the present disclosure is that it provides a cost-effective and efficient process for providing a supplement directly from biological sources.

Still another advantage of the subject matter of the present disclosure is that is teaches that commercially available food products, such as animal broths, can be transformed into dietary supplements that improve epithelial cell membrane integrity and barrier functions.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a schematic of a tight junction.

FIG. 2 is a schematic overview of a TEER experimental design for measuring the integrity of Caco-2 epithelial membranes.

FIG. 3 is flow chart illustrating an example of a process for preparing a dietary supplement according to the present disclosure.

FIG. 4 is a flow chart of another example of a process for preparing a dietary supplement according to the present disclosure.

FIG. 5 is a graph of TEER data versus time showing the effects of a broth derived supplement on tight junctions in TEER assays.

FIG. 6 is a graph of TEER data versus time showing the effects of a gizzard derived supplement on tight junctions in TEER assays.

FIG. 7 is a bar graph denoting the ability of two chicken broth preparations to induce increased cell proliferation.

### DETAILED DESCRIPTION OF THE INVENTION

The supplements and methods described herein are based on the premise that unimpeded wound healing occurs when there is a full complement of both growth factors and wound healing proteins available at concentrations, and in a format, that favors immediate recruitment. Such composition can be achieved by: careful selection of proper starting material; a gentle disruptive hydrolysis, or other isolation or extraction technique to form extracted agents; selective transformation of the extracted agents with ethanol to form transformed agents; and sequestration of the active transformed agents by phase separation techniques, or their equivalents; and the formation of a dietary supplement using the alcohol soluble components of the transformed agents.

Specifically, the present supplement compositions can be derived from natural sources. The natural sources may comprise an unpurified digest of animal tissue containing a complex mixture of proteins, peptides, growth factors, and nutrients associated with healthy tight junctions and homeostasis. The natural sources may be carefully selected for their healing characteristics. An example of a natural source is smooth animal muscle tissue, such as, but not limited to, stomachs, livers, and gizzards, from such as chicken, bovine, krill, sea urchin, and others.

In an example, the starting material is chicken gizzards, which are minced, and boiled in water for approximately 10-20 hours (e.g., 12 hours) to produce a chicken broth. Alternatively, the starting material can be commercially available meat broth (e.g., chicken broth, beef broth, among others). Other equivalent starting materials will be apparent to those skilled in the art based on the teachings provided herein.

When using an animal broth source material, whether commercially available or otherwise, the animal broth can be treated with a transforming vehicle, such as, but not limited to, ethanol. The amount of transforming vehicle used can be varied to achieve optimal levels of enrichment to form the active composition. In an example, the transforming vehicle can be used to convert an otherwise non-reactive broth into a formulation that positively affects tight junctions in an in vitro assay. Transformation can be visually monitored and/or monitored by light microscopy, and/or monitored by measuring changes in optical density spectrophotometrically.

The alcohol soluble solution (including the active agents) can be decanted, wherein the formed precipitants are removed from the active agent solution. The active agent rich fractions can be combined based on desirable qualities. Following the removal of the transforming vehicle by simmering under low heat, the active agent solution can be selectively harvested by phase separation processes and processed into the supplement for delivery (e.g. concentrated solution, dried to a powder, converted to an elixir, among others).

In an example, the method of forming the composition can include combining the animal source (e.g., chicken gizzards) and distilled or deionized water in a 1:1 ratio in any suitable processing equipment intended to sufficiently reduce particle size to yield a smooth paste free of visible intact pieces. The paste can then be heated between 75° C. and 95° C. in a closed system capable of continuous mixing for a time period between 9 and 18 hours. The mixture can then be removed from the heat source and the liquid can be separated from any solids (e.g., precipitates) by centrifugation, filtration, and/or decanting, among other separation methods. The collected liquid can be combined with food-grade ethanol in proportions ranging from 1:1 to 1:3 parts liquid to ethanol. The ethanol and collected liquid mixture can be maintained at a temperature between 2° C. to 6° C. for a time period between 8 and 120 hours, during which time a precipitate forms in the mixture. The precipitate can be removed by any conventional means including, but not limited to, centrifugation, filtration, vacuum filtration, decanting, among others. The collected liquid free of precipitate can then be further purified to remove the ethanol from the solution. The ethanol can be removed by employing any suitable process and/or processing equipment including, but not limited to, steam evaporation, rotary evaporation, heating the solution, among others. After the ethanol is removed, the solution can be further concentrated by suitable means in order to obtain a condensed active solution suitable for use. For example, the dilute solution can be dried via any common drying process that reduces the moisture level to a point that the dried invention is shelf-stable (e.g., 0.1% to 3.0% moisture). Suitable drying processes include, but are not limited to, freeze-drying, spray drying, agglomeration, drum drying, among others.

The supplement composition can include macronutrients, such as fats, carbohydrates, proteins, and fiber components. For example, the extracted agents from the source can include glutamine, phytochemicals, carotenoids, vitamin E, vitamin C, zinc, selenium, or combinations thereof.

The supplement composition can include various viscous fibers, such as, but not limited to guar gum, locust bean gum, xanthan gum, that may demonstrate an improved efficacy of the invention by forming a viscous ‘bolus’ that moves more slowly through the gut lumen than the untreated patient, thus increasing contact time with injured internal epithelial cells and allowing for increased efficacy in protecting, repairing, restoring, and maintaining healthy epithelial cells.

All materials, chemicals, and procedures used in the manufacture of the supplements taught herein may be “food safe” certified (e.g., designated as generally recognized as safe (GRAS) by the American Food and Drug Administration). The disclosed methods include selecting a source as a starting material (e.g. animal muscle tissue) that is minced into small pieces, converted into a puree (or smooth paste), or subject to any other particle reduction mechanism that increases the surface area of the starting material to then promote the release of the agent by a gentle heat or enzyme digestion. The soluble extracted agent can be collected by selective phase precipitation, filtration, or electrochemical or chromatographic methodologies.

The delivery method of the supplement comprising the active agents depends on the specific application and biological efficacy desired. For example, following removal of the transforming vehicle, the active agents can be incorporated into a supplement that can be administered directly as a food, candy, drink, mouthwash, among others. For example, the composition can be included in a dilute liquid form that may be utilized as a base for liquids such as hot or cold beverages (carbonated and/or still), soups, broths, syrups, juice, vegetable extracts, caffeinated beverages, among others. The composition can include flavor, color, among other additives. In addition, the supplement can be included in or as food additives, gummies, chewing gum, candy, lozenges, lollipops, antacid formulations, or hydrogels. For example, the composition can be included in a formula using a liquid ingredient for the purpose of hydrating a powdered matrix, such as flours used in the manufacture of pastas, baked goods, snack foods, among many others. Alternatively, the composition can be provided in a powder form that can be incorporated in recipes using dry ingredients that may be post-processed in any manner of methods specific to the end product. For example, the dry form of the composition can be used in shelf-stable prepared meals, such as macaroni and cheese, among others. The supplement can include a timed-released coatings, hydrogels, or carriers that can modulate bioavailability. The formulation can also be combined with other identified agents that can promote GI tract health, like probiotics.

The composition can be formed into a supplement prepared using methods known to the skilled person. For example, alcohol soluble components (i.e., the composition solution free of participates) can be encapsulated at appropriate dosage concentrations with various substances conventionally used as gelling agents or thickening agents in the field of food products. Examples of gelling agents include agar, gellan gum, carrageenan, pectin, pre-gelatinized starch, modified starch and gelatin. Examples of thickening agents include furcellaran, locust bean gum, guar gum, gum Arabic and xanthan gum. These gelling agents and thickening agents can be used singly or in combination. The combination of a gelling agent and a thickening agent is especially preferable. Gelling agents and/or thickening agents exhibit an appropriate gelling ability and gel stabilizing ability and control the gel strength of the resulting gel.

The supplement can be a nutritional supplement, which can be administered to the patient in a single dose, via multiple doses over a course of treatment, or on a routine ongoing basis. For example, the supplement can be administered in a single dose once, twice, or three times per day. In another example, the supplement may be administered up to five times per day. In still other examples, the supplement may be administered between three to seven times per day. For example, the supplement can be part of a vitamin regimen or probiotic supplement.

The supplement can include a flavor to enhance its palatability, especially in a pediatric population. Artificial sweeteners may be added to complement the flavor and mask the salty taste. Useful artificial sweeteners include saccharin, NutraSweet, sucra lose, acesulfane-K (ace-K), etc.

Preservatives may be added to help extend shelf life of the supplement. Persons knowledgeable in the art will he able to select the appropriate preservative, in the proper amount, to accomplish this result. Typical preservatives include, but are not limited to, potassium sorbate and sodium benzoate.

In addition, the supplement can include suitable carbohydrates, lipids and proteins as are known to those skilled in the art of making nutritional formulas. Suitable carbohydrates include, but are not limited to, hydrolyzed, intact, naturally and/or chemically modified starches sourced from corn, tapioca, rice or potato in waxy or non-waxy forms; and sugars such as glucose, fructose, lactose, sucrose, maltose, high fructose corn syrup, corn syrup solids, fructo-oligosaccharides, and mixtures thereof. Maltodextrins are polysaccharides obtained from the acid or enzyme hydrolysis of starches (such as those from corn or rice).

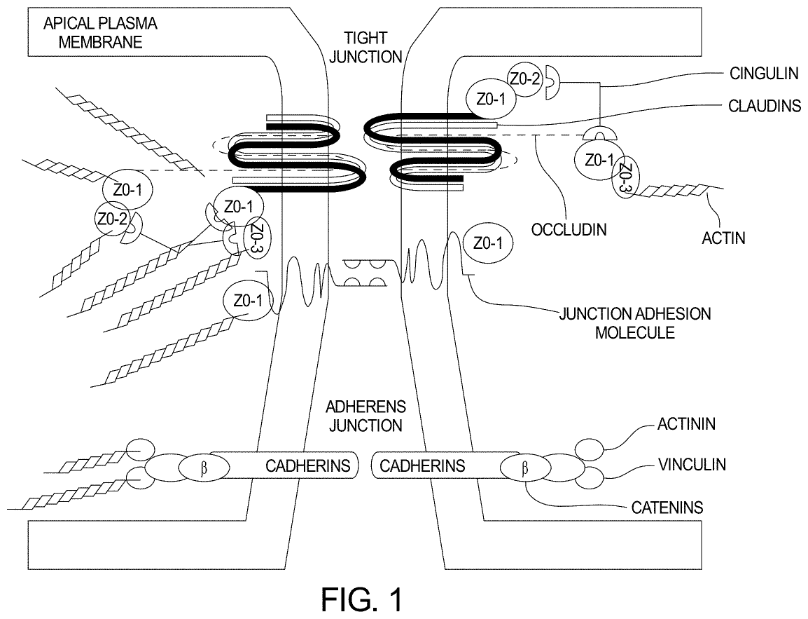

FIG. 1 is a schematic of an example of a tight junction. Differentiated human Caco-2 cells are a useful model of the intestinal barrier with functional tight junction complexes. Caco-2 cells can form an artificial epithelial cell membrane that can be challenged with nutrients and toxins. Integrity of this membrane can be monitored with a TEER assay format and agents can be screened for their ability to improve or destroy intestinal barrier function. A metric of enhanced protection of barrier function is the achievement of a level of TEER values 100% or higher than the control.

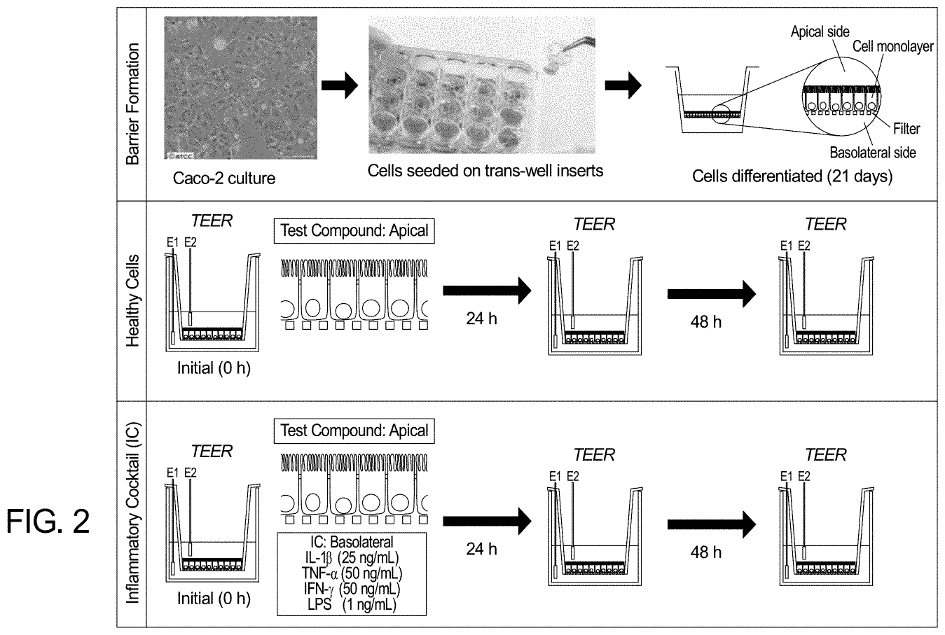

For example, FIG. 2 shows an overview of a TEER experimental design for measuring the integrity of Caco-2 epithelial membranes. In the example shown in FIG. 2, trans-epithelial electrical resistance (TEER) readings are performed at discreet, designated time points (e.g., 0, 28, and 48 hours, etc.) using an epithelial voltohmmeter, such as the one sold under the trademark name EVOM2 by World Precision Instruments, or continuously (e.g., for 24 continuous hours, for 48 continuous hours, etc.) or at hourly intervals using a cell monitoring system, such as the one sold under the trademark name CellZscope by nanoAnalytics GmbH. Higher potentials correspond to greater membrane integrity. The lower the potential, the more disruption of the membrane.

In the example shown, the experimental design tests food grade samples for effects on intestinal barrier function in cultured intestinal cells. Caco-2 cells are cultured on plates (such as those sold under the trademark name Transwell by Corning Incorporated) and cultured for 21 days to form a model intestinal barrier. Subsequently, cells are treated with test compounds, with or without an inflammatory stimulus (inflammatory cocktail, IC), composed of proinflammatory cytokines and lipopolysaccharaide (LPS). The inflammatory cocktail treatment induces intestinal barrier dysfunction. Test compounds that prevent IC-induced barrier dysfunction may have anti-inflammatory cytokines and lipopolysaccharide (LPS). Test compounds that prevent IC-induced barrier dysfunction may have anti-inflammatory and/or barrier-promoting activities. Test compounds that increase barrier function in healthy cells likely have barrier-promoting activities. In the example shown, genistein, a soy isoflavone, is used as a positive control. Barrier function is determined by trans-epithelial electrical resistance (TEER) and reported and described in Equations 1 and 2.

TEER (Δ% Initial)=100*(TEER_{24 or 28 hours}−TEER_{0 hour})/TEER_{0 hour} (1)

TEER (Δ% IC Normalized)=100*TEER (Δ% Initial)ₛₐₘₚₗₑ/TEER (Δ% Initial)_{IC} (2)

On day 20 of growth, the EVOM2 voltohmmeter is prepared by plugging it in and placing the “chopstick” electrode in 0.1 M KOH. On day 21, the cultured Caco-2 cell samples are processed as follows, or equivalent.

For example, 24-well plates containing membrane inserts with Caco-2 cells are removed from a CO2 incubator (37° C., 5% CO2, ˜91% Relative Humidity) and moved into a biosafety hood. The modified growth media is removed from both the basal and apical layers of the cell inserts. The cells are washed with sterile phosphate buffered solution and a Hanks Balanced Salt Solution (HBSS) is added to the apical (0.2 ml) and basal layers (1 ml). The 24-well plates are returned to the CO2 incubator and allowed to incubate for 30 minutes. A hotplate in the biosafety hood is set to 37° C. and the EVOM2, along with the chopstick electrodes, are moved to the biosafety hood. The chopstick electrodes are submerged in 70% ethanol solution for 15 minutes and then held in the HBSS. At 30 minutes, each plate is removed from the incubator one by one and placed on the hot plate. The covers are removed and, using the “chopstick” electrodes, each insert is measured in triplicate. The average of the three measurements establishes a baseline for each insert. When all the inserts have been measured, the plate is returned to the hood, and the next plate is measured. Once all the plates have been measured, they are brought into the biosafety hood again for the introduction of experimental media and inflammatory cocktail at time T=0.

At time T=0, the HBSS is removed from both the apical and basal compartments and replaced with inflammatory cocktail (or experimental growth media, in the case of positive controls) and experimental media in the apical layer according to the experimental design. The plates are returned to the CO2 incubator for 24-hours. At 24-hours, the plates are removed from the incubator and the measurements are repeated. In instances in which 48 hour measurements are taken, another set of measurements are taken at T=48 hours. Once the final measurements are taken, the cells are terminated using a 10% bleach solution and disposed of according to biosafety hazard protocols.

In this example, dry samples are prepared by weighing out 10 mg of material into 15 mL conical centrifuge tubes, re-suspending dry material in modified DMEM growth media, vortexing at 2000 RPM for 30 seconds, and sonicating at 37° C. for 20 minutes. The solutions are then filtered using a 0.2-micron syringe tip filter. This stock solution is then used to create samples of the desired concentration by diluting the sterile stock solutions further with the modified growth media.

In this example, liquid samples are prepared by thawing liquid samples in a hot water bath at 37° C. for about 5 minutes or until fully thawed, adding the volume of supplied liquid to modified DMEM growth media, and filtering the solutions using a 0.2-micron syringe tip filter. This stock solution is then used to create samples of the desired concentration by diluting the sterile stock solutions further with the modified growth media.

In the example shown, the inflammatory cocktail is prepared by the following process: concentrated stock solutions of the necessary cytokines are stored in the −80° C. freezer and properly aliquoted to minimize degradation from thaw-freeze cycles. The cytokine aliquots are removed from the freezer and brought into the hood for processing. The cytokines are added to the modified growth media to arrive at the final concentration of 50 ng/ml (TNF-α), 25 ng/ml (IL-1β), 50 ng/ml (IFN-γ), 1 μg/ml (Lipopolysaccharide).

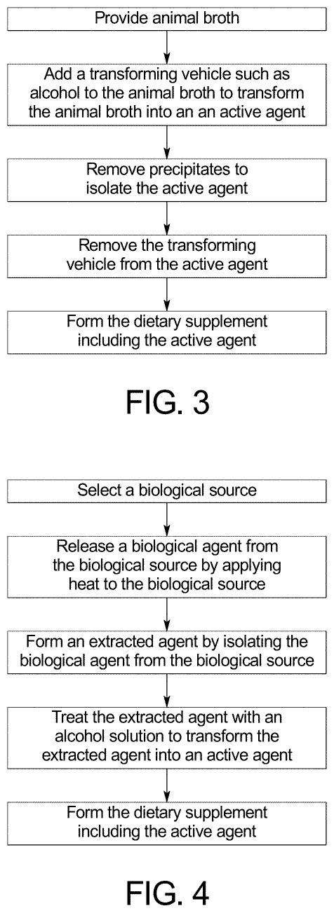

FIGS. 3 and 4 illustrate examples of processes for preparing dietary supplements according to the teachings presented herein. These examples are illustrative of the subject matter of this disclosure.

FIG. 3 illustrates an example of a process for preparing a dietary supplement for improving gastrointestinal functionality, wherein the method comprises the steps of: providing an animal broth; transforming the animal broth into an active agent by adding a transforming vehicle to the animal broth, wherein the transforming vehicle is an alcohol; isolating an active agent by removing precipitates; removing the transforming vehicle from the active agent; and forming the dietary supplement, wherein the dietary supplement includes the active agent. In some examples of the method shown in FIG. 3, the alcohol is ethanol.

In an example of the process illustrated in FIG. 3, the process for preparing a dietary supplement for improving gastrointestinal functionality can include selecting an animal broth. The selected animal broth may be a commercially available chicken broth such as, for example, the animal broth sold under the trademark name Swanson Unsalted Chicken Broth by CSC Brand LP. In other examples, the animal broth may be a different chicken broth or may be an animal broth other than a chicken broth, for example, a beef broth. Suitable animal broths will be recognized by those skilled in the art based on the practice of the inventions described herein.

In an example of the process illustrated in FIG. 3, the step of transforming the animal broth into an active agent by adding a transforming vehicle to the animal broth may include combining the animal broth with food-grade ethanol in proportions ranging from 1:1 to 1:3 parts liquid to ethanol. The ethanol and collected liquid mixture can be maintained at a temperature between 2° C. to 6° C. for a time period between 8 and 120 hours, during which time a precipitate forms in the mixture.

In an example of the process illustrated in FIG. 3, the step of isolating an active agent by removing precipitates can be accomplished by any conventional means including, but not limited to, centrifugation, filtration, vacuum filtration, decanting, among others.

In an example of the process illustrated in FIG. 3, the step of removing the transforming vehicle from the active agent can include further purification of the active agent by removing the ethanol from the solution. The ethanol can be removed by employing any suitable process and/or processing equipment including, but not limited to, steam evaporation, rotary evaporation, heating the solution, among others.

After the ethanol is removed the step of removing the transforming vehicle from the active agent can additionally include further concentration of the active agent in order to obtain a condensed active agent suitable for use. For example, the dilute solution can be dried via any common drying process that reduces the moisture level to a point that the dried invention is shelf-stable (e.g., 0.1% to 3.0% moisture). Suitable drying processes include, but are not limited to, freeze-drying, spray drying, agglomeration, drum drying, among others.

In an example of the process illustrated in FIG. 3, the step of forming the dietary supplement, wherein the dietary supplement includes the active agent, may include, as described in detail above, incorporating the active agent into a supplement that can be administered directly as an edible supplement in the form of a food, candy, drink, mouthwash, etc. Additional details as to the processes and compositions are included above in this detailed description.

FIG. 4 illustrates an example of a process for preparing a dietary supplement for improving gastrointestinal functionality, wherein the method comprises the steps of: selecting a biological source; applying heat to the biological source to release a biological agent from the biological source; isolating the biological agent from the biological source to form an extracted agent; transforming the extracted agent into an active agent by treating the extracted agent with an alcohol solution; and forming the dietary supplement, wherein the dietary supplement includes the active agent.

In an example of the process illustrated in FIG. 4, the process for selecting a biological source may include selecting chicken gizzards as a biological source. In alternative examples, another animal tissue may be used as the biological source.

In an example of the process illustrated in FIG. 4, the process for applying heat to the biological source to release a biological agent from the biological source may include mincing the chicken gizzards (or other animal tissue), and then boiling them in water for approximately 10-20 hours (e.g., 12 hours) to produce a chicken broth. For example, chicken gizzards can be combined with distilled or deionized water in a 1:1 ratio in any suitable processing equipment intended to sufficiently reduce particle size to yield a smooth paste (or puree) free of visible intact pieces. The paste can then be heated between 75° C. and 95° C. in a closed system capable of continuous mixing for a time period between 9 and 18 hours.

In an example of the process illustrated in FIG. 4, isolating the biological agent from the biological source to form an extracted agent may include removing the heated biological source from the heat source and separating liquid from any solids (e.g., precipitates) by centrifugation, filtration, and/or decanting, among other separation methods. The separated liquid is the extracted agent.

In an example of the process illustrated in FIG. 4, transforming the extracted agent into an active agent by treating the extracted agent with an alcohol solution includes combining the extracted agent (i.e., the separated liquid) with food-grade ethanol in proportions ranging from 1:1 to 1:3 parts liquid to ethanol. The ethanol and liquid mixture can be maintained at a temperature between 2° C. to 6° C. for a time period between 8 and 120 hours, during which time a precipitate forms in the mixture. The precipitate can be removed by any conventional means including, but not limited to, centrifugation, filtration, vacuum filtration, decanting, among others. The collected liquid free of precipitate can then be further purified to remove the ethanol from the solution. The ethanol can be removed by employing any suitable process and/or processing equipment including, but not limited to, steam evaporation, rotary evaporation, heating the solution, among others. After the ethanol is removed, the solution can be further concentrated by suitable means in order to obtain a condensed active solution suitable for use. For example, the dilute solution can be dried via any common drying process that reduces the moisture level to a point that the dried invention is shelf-stable (e.g., 0.1% to 3.0% moisture). Suitable drying processes include, but are not limited to, freeze-drying, spray drying, agglomeration, drum drying, among others.

In an example of the process illustrated in FIG. 4, forming the dietary supplement, wherein the dietary supplement includes the active agent, includes, as described in detail above, incorporating the active agent into a supplement that can be administered directly as an edible supplement in the form of a food, candy, drink, mouthwash, etc. Additional details as to the processes and compositions are included above in this detailed description.

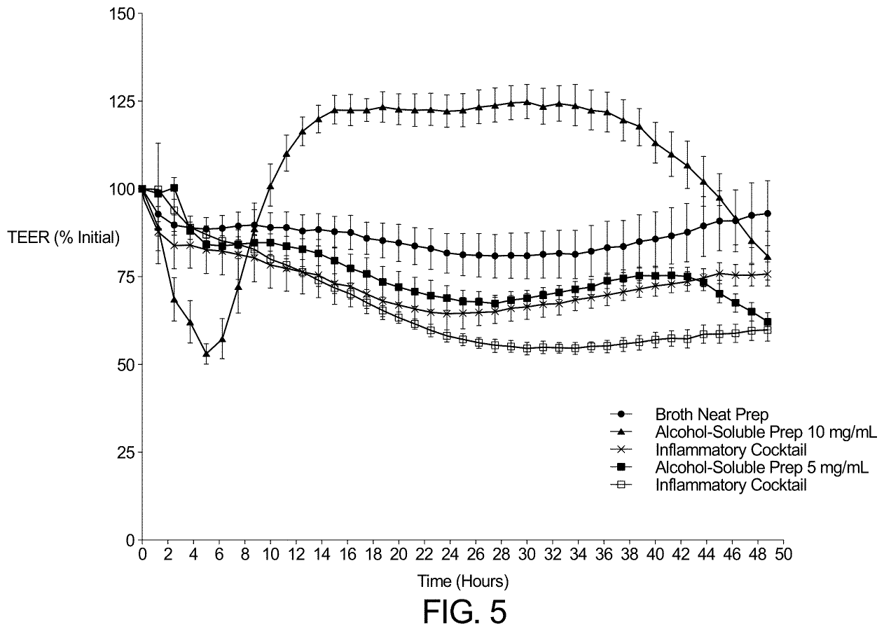

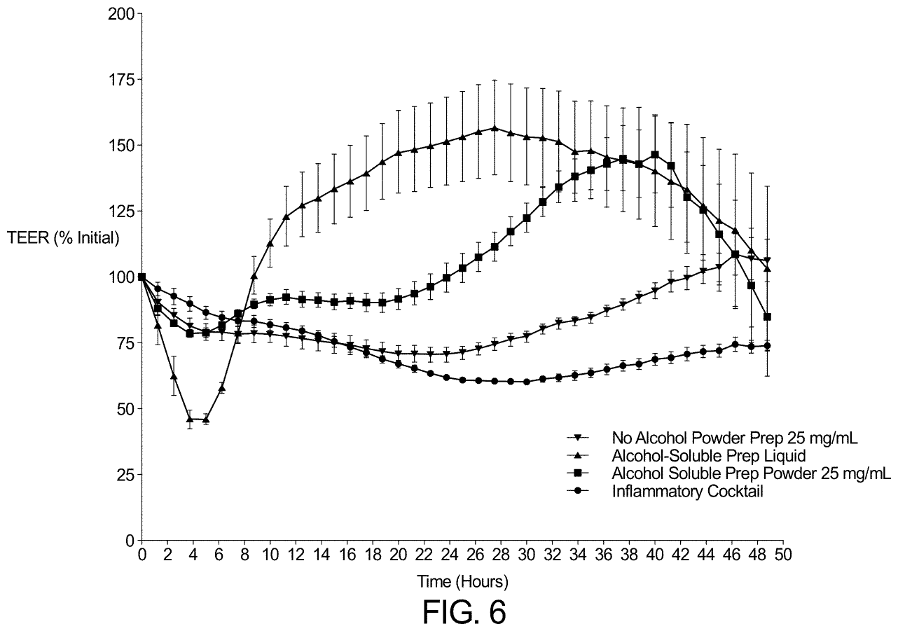

Turning now to experimental results obtained when testing the subject matter presented herein, FIG. 5 is a graph of TEER data versus time showing the effects of a broth derived supplement on tight junctions in TEER assays. Similarly, FIG. 6 is a graph of TEER data versus time showing the effects of a gizzard derived supplement on tight junctions in TEER assays.

The results shown in FIGS. 5 and 6 are representative of results obtained by following the TEER experimental design for measuring the integrity of Caco-2 epithelial membranes described above with respect to FIG. 2. As shown on the x-axis, TEER readings were taken every hour between T=0 and T=48. The y-axis represents the barrier function of the Caco-2 epithelial membranes as determined by trans-epithelial electrical resistance (TEER) plotted as TEER (Δ% Initial)=100*(TEER_{24 or 28 hours}−TEER_{0 hour})/TEER_{0 hour}. TEER readings below 100 are representative of diminished epithelial membrane integrity resulting in diminished barrier function of the epithelial membrane. TEER readings above 100 are representative of restored or enhanced epithelial membrane integrity and enhanced barrier function in the epithelial membranes.

In the example shown in FIG. 5, the experiment was run for two inflammatory cocktails, a commercial chicken broth combined with an inflammatory cocktail, a 5 mg/mL liquid preparation of the alcohol-soluble components of the commercial chicken broth combined with an inflammatory cocktail, and a 10 mg/mL liquid preparation of the alcohol-soluble components of the commercial chicken broth combined with an inflammatory cocktail.

As shown in FIG. 5, the inflammatory cocktails resulted in diminished barrier function of the epithelial membrane throughout the 48-hour testing period. Neither the untreated commercially available chicken broth, nor the 5 mg/mL liquid preparation of the alcohol-soluble components of the commercial chicken broth were able to fully counteract the effects of the inflammatory cocktail, each showing diminished barrier function of the epithelial membrane throughout the 48-hour testing period. However, the 10 mg/mL liquid preparation of the alcohol-soluble components of the commercial chicken broth showed increased barrier function throughout the majority of the 48-hour testing period. Accordingly, the results shown in FIG. 5 support the conclusion that a liquid-based dietary supplement including a concentration of approximately, or at least, 10 mg/mL of alcohol-soluble preparation derived from commercially available chicken broth may provide enhanced barrier function of tight junctions.

In the example shown in FIG. 6, the experiment was run for an inflammatory cocktail, a 25 mg/mL powder preparation of an animal broth made from chicken gizzards combined with an inflammatory cocktail, a 25 mg/mL powder preparation of the alcohol-soluble components of an animal broth made from chicken gizzards combined with an inflammatory cocktail, and a 25 mg/mL liquid preparation of the alcohol-soluble components of an animal broth made from chicken gizzards combined with an inflammatory cocktail.

As shown in FIG. 6, just as in FIG. 5, the inflammatory cocktail resulted in diminished barrier function of the epithelial membrane throughout the 48-hour testing period. The 25 mg/mL powder preparation of an animal broth made from chicken gizzards was unable to counteract the inflammatory cocktail and, therefore, showed diminished barrier function of the epithelial membrane throughout substantially all of the 48-hour testing period. However, both the 25 mg/mL powder preparation of the alcohol-soluble components of an animal broth made from chicken gizzards and the 25 mg/mL liquid preparation of the alcohol-soluble components of an animal broth made from chicken gizzards showed increased barrier function throughout a statistically relevant portion of the 48-hour testing period. Accordingly, the results shown in FIG. 6 support the conclusion that both powder-based and liquid-based dietary supplements including a concentration of approximately, or at least, 25 mg/ML of the alcohol-soluble components of an animal broth made from chicken gizzards may provide enhanced barrier function of tight junctions.

FIGS. 5 and 6 are representative examples of testing that has been performed on the subject matter presented herein. Numerous assays were completed with redundant sources and preparations demonstrating repeatable results.

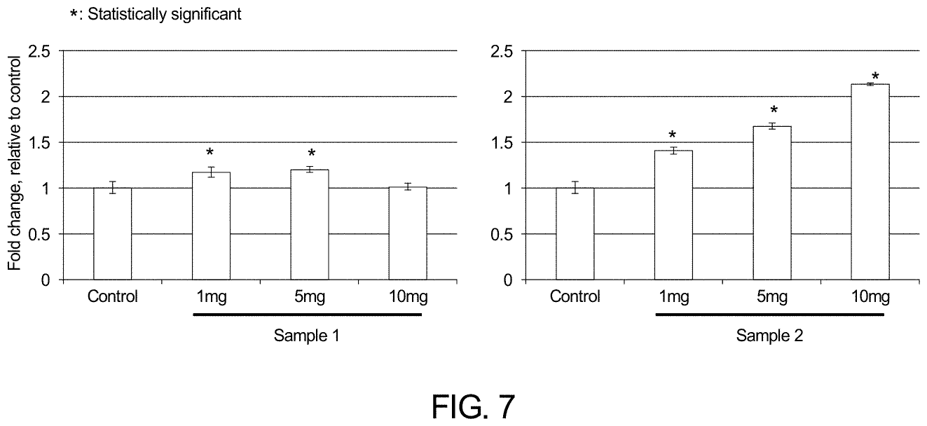

FIG. 7 illustrates a pair of bar graphs showing the cell proliferation of a Caco-2 epithelial membrane when subjected to various compositions. In the example shown in FIG. 7, mitogenesis was assayed by performing cell counts four days after exposing a confluent culture of indicator cells to the agent of interest, adding trypsin to prepare a suspension of single cells, and confirming cell separation while counting them in a hemocytometer. Mitogenic activity was assessed in each of the following preparations: chicken gizzard derived supplement using no alcohol extraction step (Sample 1), and chicken broth derived supplement that utilized an alcohol treatment step (Sample 2).

Although both compositions caused an increase in cell proliferation, in an in vitro assay, as shown, the compositions of ethanol-treated commercial chicken broth (Sample 2) demonstrated superior cell proliferation relative to the control. Moreover, the compositions of ethanol-treated commercial chicken broth (Sample 2) further demonstrated a dose dependency, with the higher concentrations showing greater cell proliferation. Greater cell proliferation represents a restoration and/or enhancement of the barrier function of the epithelial membrane and is suggestive of wound repair and healthy tight junctions.

Accordingly, the subject matter provided herein supports a conclusion that, in the appropriate concentration, the alcohol-soluble components of both “off-the-shelf” commercially available animal broths, as well as animal broths developed from animal tissue such as chicken gizzards, demonstrate the ability to restore and/or improve epithelial membrane integrity and barrier function.

In experiments related to those represented by FIGS. 5 and 6, TEER data demonstrated the effects of residual ethanol in the broth digests. When residual ethanol was present (i.e., not completely boiled off from the active agent solution), TEER measurements were lower than the inflammatory cocktail tracings. Accordingly, this data demonstrates that there are advantages to completely removing any residual ethanol in the broth digests.

It should be noted that various changes and modifications to the presently preferred embodiments described herein will be apparent to those skilled in the art. Such changes and modifications may be made without departing from the spirit and scope of the present invention and without diminishing its attendant advantages.

We claim:

1. A method for preparing a dietary supplement, the method comprising:
providing a biological agent;
transforming the biological agent into an active agent by treating the biological agent with an alcohol to form a precipitant and a supernatant, wherein the supernatant includes alcohol soluble components; and
forming the dietary supplement using the alcohol soluble components.

2. The method of claim 1, wherein the transforming step includes mixing the biological agent with the alcohol.

3. The method of claim 1, wherein the transforming step comprises adding the alcohol to the active agent at a ratio between 1:1 and 1:3 parts biological agent to alcohol by volume.

4. The method of claim 3, wherein the alcohol and the active agent are maintained at a temperature of between 2° C. to 6° C. for a duration of between about 8 hours and about 120 hours.

5. The method of claim 1, further comprising:
decanting the supernatant from the precipitant; and
concentrating the supernatant to form the active agent.

6. The method of claim 1, wherein treating the biological agent with the alcohol is configured to cause precipitation of insoluble components.

7. The method of claim 6, wherein the transforming step further comprises removing the insoluble components by one of centrifugation, filtration, vacuum filtration, or decanting.

8. The method of claim 1, further comprising the step of removing any remaining alcohol from the active agent through one of steam evaporation, rotary evaporation, or heating.

9. The method of claim 8, further comprising the step of drying the active agent through one of freeze drying, spray drying, agglomeration, or drum drying.

10. The method of claim 1, wherein the biological agent comprises one of an animal broth, animal gizzards, or animal tissue.

11. The method of claim 1, wherein the alcohol includes ethanol.

12. The method of claim 1, further comprising the step of incorporating the dietary supplement into one of a food product, a hydrogel, a tablet, a lozenge, a vitamin, a powder, a liquid, a candy, and a gel.

13. A method for preparing a dietary supplement, wherein the method comprises the steps of:
transforming an animal broth into an active agent by adding a transforming vehicle to the animal broth, wherein the transforming vehicle is an alcohol;
isolating the active agent by removing precipitates;
removing the transforming vehicle from the active agent; and
forming the dietary supplement, wherein the dietary supplement includes the active agent.

14. The method of claim 13, wherein the step of transforming the animal broth into the active agent by adding the transforming vehicle to the animal broth includes combining the animal broth with an ethanol solution to form a supernatant, decanting the supernatant, and concentrating the supernatant to form the active agent.

15. The method of claim 13, further comprising the step of, after transforming the animal broth into the active agent, heating the active agent to remove a portion of the alcohol.

16. The method of claim 13, further comprising incorporating the dietary supplement into at least one of a food product, hydrogel, solution, tablet, lozenge, vitamin, powder, candy, and gel.

17. The method of claim 13, wherein the step of transforming the animal broth into the active agent by adding the transforming vehicle to the animal broth includes adding the alcohol to the animal broth in a 1:1 ratio by volume.
